(19) United States
(12) Patent Application Publication
(10) Pub. No.: US 2026/0346584 A1
(43) Pub. Date: Nov. 5, 2026

(54) DECORATIVE SCAR COVER PRODUCTS AND RELATED METHODS

(71) Applicant: Scarbright Holdings Inc., Brooklyn, NY (US)

(72) Inventor: Michael Rothman, Brooklyn, NY (US)

(73) Assignee: Scarbright Holdings Inc., Brooklyn, NY (US)

(21) Appl. No.: 19/478,418

(22) PCT Filed: Apr. 26, 2024
(86) PCT No.: PCT/US2024/026674

§ 371 Date: Oct. 24, 2025

Related U.S. Application Data
(60) Provisional application No. 63/462,448, filed on Apr. 27, 2023.

(51) Int. Cl. B32B 25/20; A61F 2/50; A61F 2/78; B32B 7/12; B32B 25/02; B32B 25/04

(52) U.S. Cl. CPC B32B 25/20; A61F 2/78; B32B 7/12; B32B 25/02; B32B 25/042; A61F 2002/5001; A61F 2002/7806; B32B 2250/02; B32B 2307/41; B32B 2535/00

(57) ABSTRACT

Decorative scar cover products and related articles and methods are generally described. In some embodiments, the article comprises a first layer comprising an adhesive silicone and a second layer comprising a non-adhesive silicone disposed on the first layer, wherein the second layer comprise one or more pigments such that the second layer is impermeable to ultraviolet (UV) light. In further embodiments, a method of scar treatment is described. In certain embodiments, the method comprises applying an adhesive to a scar site, wherein the adhesive comprises a first layer comprising an adhesive silicone and a second layer comprising a non-adhesive silicone disposed on the first layer, wherein the second layer comprises one or more pigments such that the second layer is impermeable to UV light.

### RELATED APPLICATIONS

This application claims priority under 35 U.S.C. § 119(e) to U.S. Provisional Patent Application No. 63/462,448, filed Apr. 27, 2023, and entitled “DECORATIVE SCAR COVER PRODUCTS AND RELATED METHODS”, which is incorporated herein by reference in its entirety for all purposes.

### TECHNICAL FIELD

Decorative scar cover products and related articles and methods are generally described.

### BACKGROUND

Conventional scar cover products present a dull branding and packing experience that caters to an over the counter (OTC) drug store environment. Accordingly, improved scar cover products and related articles and methods are needed.

### SUMMARY

Decorative scar cover products and related articles and methods are generally described.

According to certain embodiments, an article is described. In some embodiments, the article comprises a first layer comprising an adhesive silicone and a second layer comprising a non-adhesive silicone disposed on the first layer, wherein the second layer comprise one or more pigments such that the second layer is impermeable to ultraviolet (UV) light.

According to some embodiments, a method of scar treatment is described. In certain embodiments, the method comprises applying an adhesive to a scar site, wherein the adhesive comprises a first layer comprising an adhesive silicone and a second layer comprising a non-adhesive silicone disposed on the first layer, wherein the second layer comprises one or more pigments such that the second layer is impermeable to UV light. In some embodiments, applying the adhesive to the scar site comprises applying at least portion of the first layer comprising the adhesive silicone to the scar site such that the adhesive silicone contacts the scar site. In certain embodiments, the method comprises allowing the adhesive to adhere to the scar site.

According to certain embodiments, an article is described, the article comprising a first cover, a second cover, and a plurality of pages between the first cover and the second cover. In some embodiments, at least one first page of the plurality of pages comprises at least one decorative scar cover product disposed on a release liner, wherein the at least one decorative scar cover product comprises a first layer comprising an adhesive silicone and a second layer comprising a non-adhesive silicone disposed on the first layer. In certain embodiments, the first layer and/or the second layer comprise one or more pigments. In some embodiments, at least one second page of the plurality of pages comprises a storage mechanism configured to store the decorative scar cover product.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 shows a cross-sectional schematic diagram of an article comprising a first layer and a second layer, in accordance with certain embodiments.

FIG. 2 shows a cross-sectional schematic diagram of an article comprising a first layer, a second layer, and a third layer, in accordance with certain embodiments.

FIG. 3A shows a top-view schematic diagram of an article, in accordance with certain embodiments.

FIG. 3B shows a top-view schematic diagram of an article comprising a first layer and a second layer comprising a serpentine shape, in accordance with certain embodiments.

FIG. 4A shows a top-view schematic diagram of an article comprising a third layer, in accordance with certain embodiments.

FIG. 4B shows a top-view schematic diagram of an article comprising a first layer and a second layer comprising a serpentine shape, wherein the article further comprises a third layer, in accordance with certain embodiments.

FIGS. 5A-5C show perspective-view schematic diagrams of an article comprising a first cover, a second cover, and a plurality of pages between the first cover and the second cover, in accordance with certain embodiments.

FIG. 6A shows a scar cover product in sanitary wrapping, in accordance with certain embodiments.

FIG. 6B shows a scar cover product on a release liner, in accordance with certain embodiments.

FIG. 7 shows an in-situ image of a scar cover product on a healing scar of a user, in accordance with certain embodiments.

FIGS. 8A-8D show cross-sectional schematic diagrams of a process of manufacturing a release liner and a scar cover product, in accordance with certain embodiments.

FIG. 9 shows a schematic diagram of a process of packaging a scar cover product, in accordance with certain embodiments.

FIG. 10 shows an in-situ image of three scar cover products on the skin of a user, in accordance with certain embodiments.

FIG. 11 shows an image of various scar cover product shapes and sizes, in accordance with certain embodiments.

FIG. 12 shows an in-situ image of three different scar products on the skin of a user, in accordance with certain embodiments.

FIG. 13 shows a schematic diagrams of a decorative scar cover product catalogue, in accordance with certain embodiments.

FIGS. 14A-14F show schematic diagrams of decorative scar cover products having complementary, nested, and/or repeating shapes, in accordance with certain embodiments.

FIG. 15 shows a schematic diagram of a decorative scar cover product comprising a serpentine shape, in accordance with certain embodiments.

FIG. 16 shows various common surgeries and scar types, in accordance with certain embodiments.

### DETAILED DESCRIPTION

Decorative scar cover products and related articles and methods are generally described. The decorative scar cover product is, in certain embodiments, designed to celebrate a user's recovery journey while protecting and healing the user's scar line. In certain embodiments, the decorative scar cover product draws inspiration from the Japanese art of kintsugi and momentum from concurrent trends around science-backed skincare, body art, and body positivity. In accordance with some embodiments, the decorative scar cover product redefines the wound care category from a focus on concealment to honoring the experiences that give users character and strength.

According to some embodiments, the decorative scar cover product comprises silicone (e.g., medical-grade silicone) and one or more pigments (e.g., cosmetic-grade pigments). In certain embodiments, the decorative scar cover product is in the form of a sheet. According to certain embodiments, the sheet comprises a first layer (e.g., comprising an adhesive silicone) and a second layer (e.g., comprising a non-adhesive silicone) disposed on the first layer. In some embodiments, the second layer comprises the one or more pigments. As described herein in greater detail, at least the second layer is impermeable to UV light, at least in part due to the one or more pigments in the second layer.

According to certain embodiments, the decorative scar cover product advantageously provides cosmetic, clinical, and/or psychological benefits. In some embodiments, for example, the decorative scar cover product is used to improve scar size, color, and/or texture by making the scar flatter, softer, and/or less visible. In certain embodiments, the decorative scar cover product hydrates a scar site, thereby reducing redness, itchiness, and/or discomfort. In certain embodiments, the decorative scar cover product protects a scar site from UV light, thereby avoiding dangerous pigmentation. According to some embodiments, the decorative scar cover product is non-toxic and safe for sensitive skin and children. In some embodiments, the decorative scar cover product is reusable in that it can be removed, washed, and re-applied. In certain embodiments, the shape, size, and/or color of the decorative scar cover product is user customizable and can be fine-tuned depending on the shape of the scar site that the decorative scar product is to be applied to. Configuring the decorative scar cover product in this way advantageously allows a user to use the decorative scar cover product as a decorative body ornamentation, akin to a tattoo and/or piercing.

According to some embodiments, a catalogue comprising at least one decorative scar cover product is described. In certain embodiments, the catalogue comprises a first (e.g., front) cover, a second (e.g., back) cover, and a plurality of pages between the first cover and the second cover. In some embodiments, at least one first page of the plurality of pages comprises at least one decorative scar cover product disposed on a release liner. In certain embodiments, at least one second page of the plurality of pages comprises a storage mechanism configured to store the decorative scar cover product (e.g., after releasing the decorative scar cover product from the release liner, applying the decorative scar cover product to a scar site, and removing the decorative scar cover product form the scar site).

In certain embodiments, a method of scar treatment is described. According to some embodiments, for example, a decorative scar cover product is applied to a scar site such that an adhesive portion of the decorative scar cover product adheres to the scar site. While in use, the decorative scar cover product may, in certain embodiments, facilitate healing of the scar site, hydrate the scar site, and/or protect the scar site from foreign species and/or UV light. In certain embodiments, the decorative scar cover product is removed from the scar site, washed, and reapplied to the scar site.

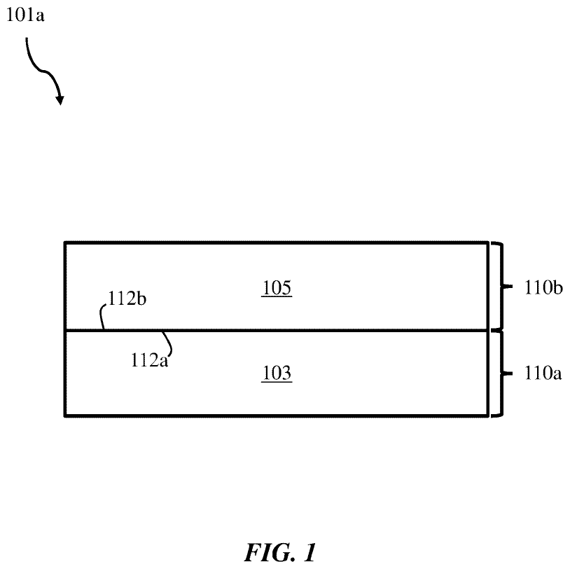

According to some embodiments, an article is described. FIG. 1 shows a cross-sectional schematic diagram of article 101a, in accordance with certain embodiments. In some embodiments, the article comprises a first layer and a second layer. Referring, for example, to FIG. 1, article 101a comprises first layer 103 and second layer 105. In certain embodiments, article 101a is a decorative scar cover product. According to some embodiments, the decorative scar cover product is in the form of a sheet. Referring, for example, to FIG. 1, article 101a is in the form of a sheet.

According to some embodiments, the first layer (e.g., first layer 103) comprises an adhesive silicone (e.g., a medical-grade adhesive silicone). The first layer may comprise the adhesive silicone in any of a variety of suitable amounts. In some embodiments, for example, the first layer comprises the adhesive silicone in an amount greater than or equal to 80 weight percent (wt. %), greater than or equal to 85 wt. %, greater than or equal to 90 wt. %, greater than or equal to 95 wt. %, greater than or equal to 98 wt. %, or greater than or equal to 99 wt. % versus a total weight of the first layer. In certain embodiments, the first layer comprises the adhesive silicone in an amount less than or equal to 100 wt. %, less than or equal to 99 wt. %, less than or equal to 98 wt. %, less than or equal to 95 wt. %, less than or equal to 90 wt. %, or less than or equal to 85 wt. % versus a total weight of the first layer. Combinations of the above recited ranges are possible (e.g., the first layer comprises the adhesive silicone in an amount greater than or equal to 80 wt. % and less than or equal to 100 wt. % versus a total weight of the first layer, the first layer comprises the adhesive silicone in an amount greater than or equal to 90 wt. % and less than or equal to 95 wt. % versus a total weight of the first layer). Other ranges are also possible.

In some embodiments, the second layer (e.g., second layer 105) comprises a non-adhesive silicone (e.g., a medical-grade non-adhesive silicone). The second layer may comprise the non-adhesive silicone in any of a variety of suitable amounts. In some embodiments, for example, the second layer comprises the non-adhesive silicone in an amount greater than or equal to 80 wt. %, greater than or equal to 85 wt. %, greater than or equal to 90 wt. %, greater than or equal to 95 wt. %, or greater than or equal to 98 wt. % versus a total weight of the second layer. In certain embodiments, the second layer comprises the non-adhesive silicone in an amount less than or equal to 99 wt. %, less than or equal to 98 wt. %, less than or equal to 95 wt. %, less than or equal to 90 wt. %, or less than or equal to 85 wt. % versus a total weight of the second layer. Combinations of the above recited ranges are possible (e.g., the second layer comprises the non-adhesive silicone in an amount greater than or equal to 80 wt. % and less than or equal to 99 wt. % versus a total weight of the second layer, the second layer comprises the non-adhesive silicone in an amount greater than or equal to 90 wt. % and less than or equal to 95 wt. % versus a total weight of the second layer). Other ranges are also possible.

In some embodiments, the first layer (e.g., first layer 103) and/or the second layer (e.g., second layer 105) comprise one or more pigments (e.g., one or more cosmetic-grade pigments). In some embodiments, the first layer comprises one or more pigments. The one or more pigments in the first layer may be evenly distributed throughout a bulk of the first layer, in accordance with some embodiments. In certain embodiments, the second layer comprises one or more pigments. In some embodiments, the one or more pigments in the second layer are evenly distributed throughout a bulk of the second layer. According to some embodiments, the first layer and the second layer comprise one or more pigments. The one or more pigments may be evenly distributed throughout a bulk of the first layer and the second layer, in accordance with certain embodiments. In some embodiments wherein the first layer and the second layer comprise one or more pigments, the one or more pigments in the first layer are different than the one or more pigments in the second layer. In other embodiments wherein the first layer and the second layer comprise one or more pigments, the one or more pigments in the first layer are the same as the one or more pigments in the second layer.

In some embodiments, the first layer (e.g., first layer 103) and/or the second layer (e.g., second layer 105) comprise a mixture of pigments (e.g., a mixture of cosmetic-grade pigments). For example, in some embodiments, the first layer and/or the second layer comprise a mixture of two pigments, a mixture of three pigments, a mixture of four pigments, a mixture of five pigments, etc. In some embodiments, the first layer comprises a mixture of pigments. The mixture of pigments may be evenly distributed throughout a bulk of the first layer, in accordance with certain embodiments. In certain embodiments, the second layer comprises a mixture of pigments. In some embodiments, the mixture of pigments are evenly distributed throughout a bulk of the second layer. According to some embodiments, the first layer and the second layer comprise a mixture of pigments. The mixture of pigments may be evenly distributed throughout a bulk of the first layer and the second layer, in accordance with some embodiments. In certain embodiments wherein the first layer and the second layer comprise a mixture of pigments, the mixture of pigments in the first layer are different than the mixture of pigments in the second layer. In other embodiments wherein the first layer and the second layer comprise a mixture of pigments, the mixture of pigments in the first layer are the same as the mixture of pigments in the second layer.

The one or more pigments may be any of a variety of suitable colors. In some embodiments, the color of the one or more pigments is gold (e.g., gold foil, kintsugi gold, rose gold), gray (e.g., gunmetal), bronze, silver, blue (e.g., electric blue), yellow, red, pink, and/or off-white. Other colors are also possible as the disclosure is not meant to be limiting in this regard. According to some embodiments, the color of the one or more pigments is intentionally different than the skin tone of a user. In other embodiments, the color of the one or more pigments is the same as the skin tone of a user. In certain embodiments wherein the first layer and/or the second layer comprise a mixture of pigments (e.g., a mixture of two pigments, a mixture of three pigments, a mixture of four pigments, a mixture of five pigments, etc.), differently colored pigments are mixed to provide unique color schemes and/or color gradients.

The first layer may comprise the one or more pigments in any of a variety of suitable amounts. In some embodiments, for example, the first layer comprises the one or more pigments in an amount greater than or equal to 0 wt. %, greater than or equal to 1 wt. %, greater than or equal to 2 wt. %, greater than or equal to 5 wt. %, greater than or equal to 10 wt. %, or greater than or equal to 15 wt. % versus a total weight of the first layer. In certain embodiments, the first layer comprises the one or more pigments in an amount less than or equal to 20 wt. %, less than or equal to 15 wt. %, less than or equal to 10 wt. %, less than or equal to 5 wt. %, less than or equal to 2 wt. %, or less than or equal to 1 wt. % based on a total weight of the first layer. Combinations of the above recited ranges are possible (e.g., the first layer comprises the one or more pigments in an amount greater than or equal to 0 wt. % and less than or equal to 20 wt. % versus a total weight of the first layer, the first layer comprises the one or more pigments in an amount greater than or equal to 5 wt. % and less than or equal to 10 wt. % versus a total weight of the first layer). Other combinations are also possible.

The second layer may comprise the one or more pigments in any of a variety of suitable amounts. In some embodiments, for example, the second layer comprises the one or more pigments in an amount greater than or equal to 1 wt. %, greater than or equal to 2 wt. %, greater than or equal to 5 wt. %, greater than or equal to 10 wt. %, or greater than or equal to 15 wt. % versus a total weight of the second layer. In certain embodiments, the second layer comprises the one or more pigments in an amount less than or equal to 20 wt. %, less than or equal to 15 wt. %, less than or equal to 10 wt. %, less than or equal to 5 wt. %, or less than or equal to 2 wt. %. Combinations of the above recited ranges are possible (e.g., the second layer comprises the one or more pigments in an amount greater than or equal to 1 wt. % and less than or equal to 20 wt. % versus a total weight of the second layer, the second layer comprises the one or more pigments in an amount greater than or equal to 5 wt. % and less than or equal to 10 wt. % versus a total weight of the second layer). Other combinations are also possible.

In certain embodiments, the second layer comprising the one or more pigments is substantially (or completely) impermeable to UV light. According to some embodiments, the one or more pigments in the second layer are advantageously configured to block UV light from passing through the second layer. In certain embodiments, for example, the one or more pigments are evenly distributed throughout a bulk of the second layer. The second layer may have any of a variety of suitable UV transmittances. As used herein, the term “UV transmittance” is given its ordinary meaning in the art and refers to the percentage of incident UV light that passes through a sample. In certain embodiments, the UV transmittance of the second layer is substantially low. In certain embodiments, the second layer has a UV transmittance less than or equal to 5%, less than or equal to 4%, less than or equal to 3%, less than or equal to 2%, less than or equal to 1%, less than or equal to 0.5%, or less than or equal to 0.1%. In some embodiments, the second layer has a UV transmittance of greater than or equal to 0%, greater than or equal to 0.1%, greater than or equal to 0.5%, greater than or equal to 1%, greater than or equal to 2%, greater than or equal to 3%, or greater than or equal to 4%. Combinations of the above recited ranges are possible (e.g., the second layer has a UV transmittance of less than or equal to 5% and greater than or equal to 0%, the second layer has a UV transmittance of less than or equal to 2% and greater than or equal to 1%). Other ranges are also possible. According to some embodiments, the UV transmittance of the second layer is measured by a spectrophotometer.

In certain embodiments, the first layer comprising one or more pigments is substantially (or completely) impermeable to UV light. The first layer may have any of the previously described UV transmittances with respect to the second layer (e.g., the first layer has a UV transmittance of less than or equal to 5% and greater than or equal to 0%).

According to some embodiments, the second layer is disposed on the first layer. Referring, for example, to FIG. 1, second layer 105 is disposed on first layer 103. In some embodiments, a surface of the first layer contacts a surface of the second layer. For example, as shown in FIG. 1, surface 112a of first layer 103 contacts surface 112b of second layer 105. In certain embodiments, the surface of the first layer is adhered to the surface of the second layer. For example, referring to FIG. 1, surface 112a of first layer 103 is adhered to surface 112b of second layer 105. According to certain embodiments, the surface of the first layer is adhered to the surface of the second layer such that the adhesive silicone of the first layer adheres to the non-adhesive silicone of the second layer.

The first layer may have any of a variety of suitable thicknesses. Referring, for example, to FIG. 1, first layer 103 has thickness 110a. In some embodiments, the thickness of the first layer is less than or equal to 10 millimeters, less than or equal to 5 millimeters, less than or equal to 1 millimeter, less than or equal to 0.5 millimeters, or less than or equal 0.2 millimeters. In certain embodiments, the thickness of the first layer is greater than or equal to 0.1 millimeters, greater than or equal to 0.2 millimeters, greater than or equal to 0.5 millimeters, greater than or equal to 1 millimeter, or greater than or equal to 5 millimeters. Combinations of the above recited ranges are possible (e.g., the thickness of the first layer is less than or equal to 10 millimeters and greater than or equal to 0.1 millimeters, the thickness of the first layer is less than or equal to 1 millimeter and greater than or equal to 0.5 millimeters). Other ranges are also possible.

The second layer may have any of a variety of suitable thicknesses. For example, referring to FIG. 1, second layer 105 has thickness 110b. In certain embodiments, the thickness of the second layer is less than or equal to 10 millimeters, less than or equal to 5 millimeters, less than or equal to 1 millimeter, less than or equal to 0.5 millimeters, or less than or equal 0.2 millimeters. In certain embodiments, the thickness of the second layer is greater than or equal to 0.1 millimeters, greater than or equal to 0.2 millimeters, greater than or equal to 0.5 millimeters, greater than or equal to 1 millimeter, or greater than or equal to 5 millimeters. Combinations of the above recited ranges are possible (e.g., the thickness of the second layer is less than or equal to 10 millimeters and greater than or equal to 0.1 millimeters, the thickness of the second layer is less than or equal to 1 millimeter and greater than or equal to 0.5 millimeters). Other ranges are also possible.

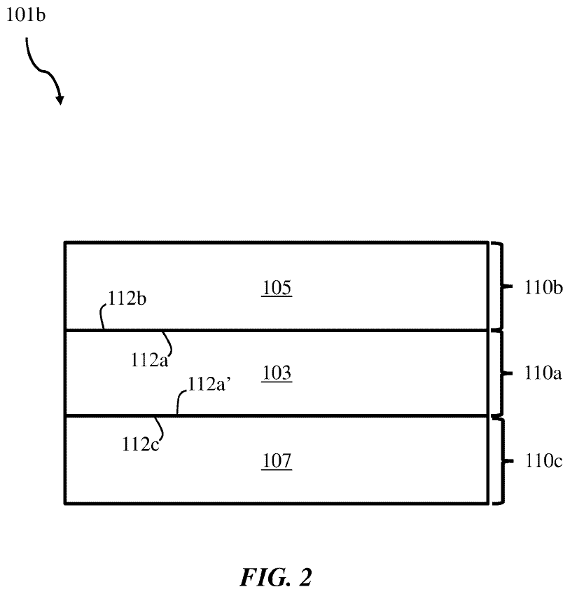

According to some embodiments, the article comprises a third layer. FIG. 2 shows a cross-sectional schematic diagram of article 101b comprising first layer 103, second layer 105, and third layer 107. In certain embodiments, article 101b comprises a decorative scar cover product (e.g., first layer 103 and second layer 105) disposed on a release liner (e.g., third layer 107).

The third layer may comprise any of a variety of suitable materials. In some embodiments, the third layer comprises paper and/or a plastic. Suitable paper materials include, but are not limited to, super calendered kraft paper, glassine, clay coated kraft paper, machine finished kraft paper, and/or machine glazed paper. Suitable plastics include, but are not limited to, biaxially oriented polyethylene terephthalate, biaxially oriented polypropylene, and/or polyethylene. Other materials are also possible as the disclosure is not meant to be limiting in this regard.

In certain embodiments, the first layer comprising the adhesive silicone is disposed on the third layer such that the adhesive silicone contacts the third layer.

Referring, for example, to FIG. 2, first layer 103 comprising the adhesive silicone is disposed on third layer 107 such that the adhesive silicone contacts third layer 107. In certain embodiments, a surface of the first layer contacts a surface of the third layer. For example, as shown in FIG. 2, surface 112a′ of first layer 103 contacts surface 112c of third layer 107. In some embodiments, the surface of the first layer is adhered to the surface of the third layer. For example, referring to FIG. 2, surface 112a′ of first layer 103 is adhered to surface 112c of third layer 107. In some embodiments, the surface of the first layer is adhered to the surface of the third layer such that adhesive silicone of the first layer adheres to the material (e.g., paper and/or plastic) of the third layer.

In some embodiments, the first layer and the second layer are configured to be released (e.g., peeled) from the third layer. Referring, for example, to FIG. 2, first layer 103 and second layer 105 are configured to be released from third layer 107. In certain embodiments, the adherence of the adhesive silicone of the first layer to the material of the third layer is weak enough to allow a user to release the first layer and the second layer from the third layer (e.g., by hand). According to some embodiments, the first layer comprising the adhesive silicone is releasably adhered to the material of the third layer. In certain embodiments, the third layer is a release liner.

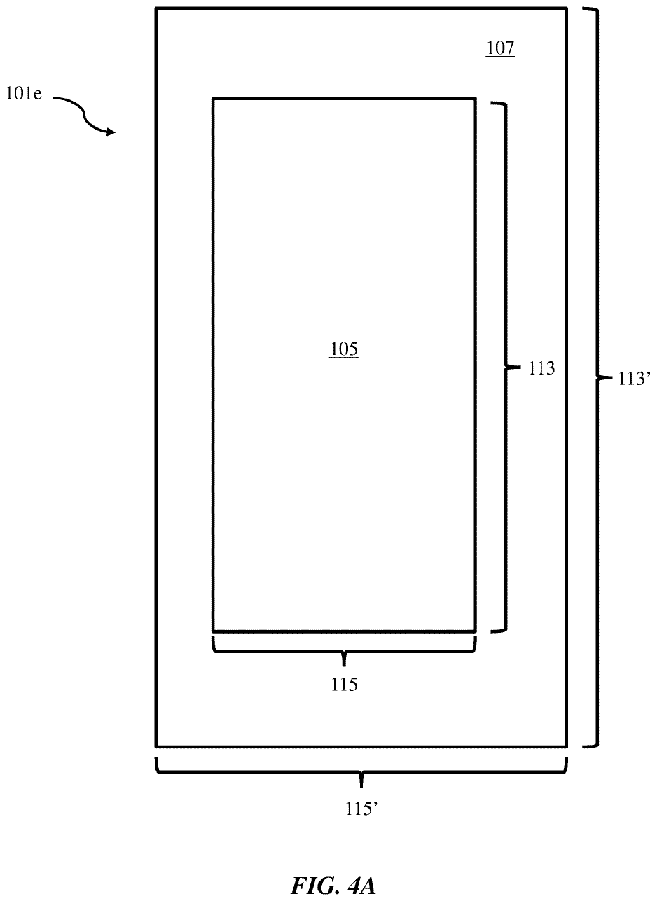

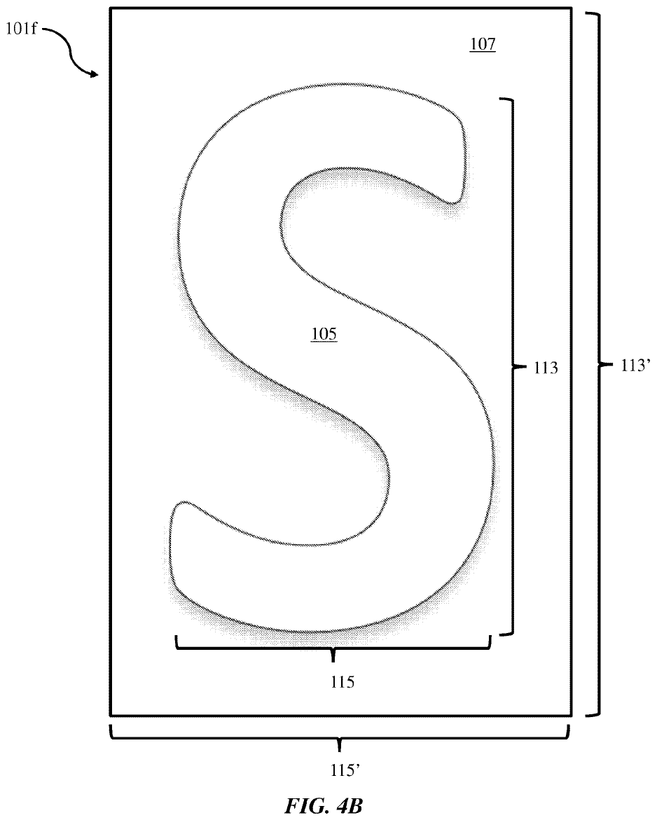

The third layer may have any of a variety of suitable thicknesses. For example, referring to FIG. 4, third layer 107 has thickness 110c. In certain embodiments, the thickness of the third layer is less than or equal to 10 millimeters, less than or equal to 5 millimeters, less than or equal to 1 millimeter, less than or equal to 0.5 millimeters, or less than or equal 0.2 millimeters. In certain embodiments, the thickness of the third layer is greater than or equal to 0.1 millimeters, greater than or equal to 0.2 millimeters, greater than or equal to 0.5 millimeters, greater than or equal to 1 millimeter, or greater than or equal to 5 millimeters. Combinations of the above recited ranges are possible (e.g., the thickness of the third layer is less than or equal to 10 millimeters and greater than or equal to 0.1 millimeters, the thickness of the third layer is less than or equal to 1 millimeter and greater than or equal to 0.5 millimeters). Other ranges are also possible.

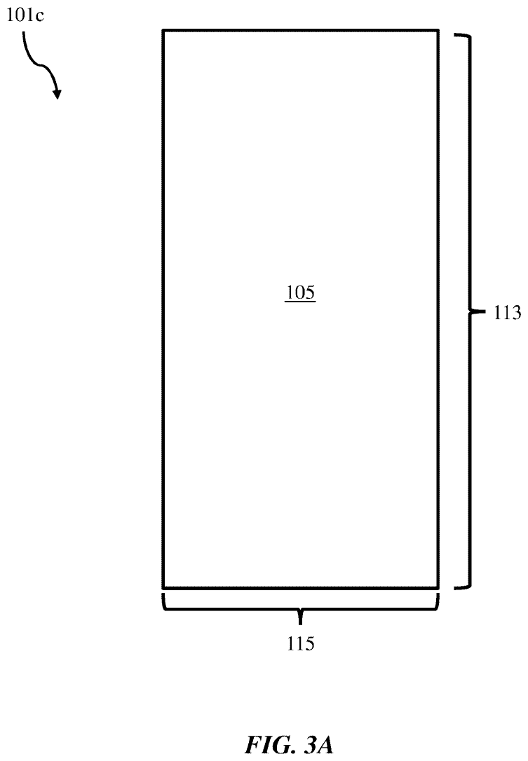

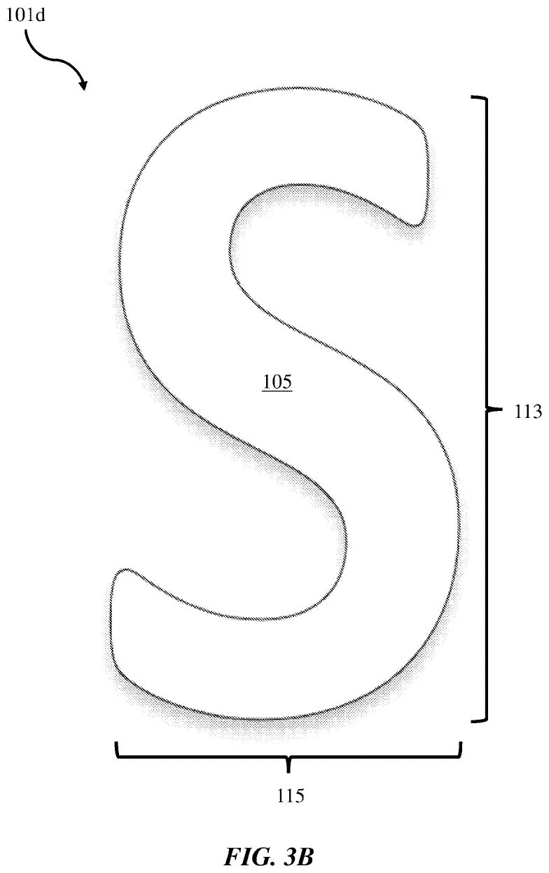

The first layer and the second layer may have any of a variety of suitable dimensions. In some embodiments, for example, the first layer and the second layer have a first dimension (e.g., a length) and a second dimension (e.g., a width) perpendicular to the first dimension. FIG. 3A shows a top-view schematic diagram of article 101c, in accordance with certain embodiments. As shown in FIG. 3A, the first layer (hidden from view in FIG. 3A by second layer 105) and second layer 105 have first dimension 113 (e.g., length) and second dimension 115 (e.g., width) perpendicular to first dimension 113.

The first dimension (e.g., the length) of the first layer and the second layer may be any of a variety of suitable values. In certain embodiments, for example, the first dimension is greater than or equal to 2 centimeters, greater than or equal to 3 centimeters, greater than or equal to 4 centimeters, greater than or equal to 5 centimeters, or greater than or equal to 10 centimeters. In certain embodiments, the first dimension is less than or equal to 20 centimeters, less than or equal to 10 centimeters, less than or equal to 5 centimeters, less than or equal to 4 centimeters, or less than or equal to 3 centimeters. Combinations of the above recited ranges are also possible (e.g., the first dimension is greater than or equal to 2 centimeters and less than or equal to 20 centimeters, the first dimension is greater than or equal to 4 centimeters and less than or equal to 5 centimeters). Other ranges are also possible.

The second dimension (e.g., the width) of the first layer and the second layer may be any of a variety of suitable values. In some embodiments, for example, the second dimension is greater than or equal to 10 centimeters, greater than or equal to 12 centimeters, greater than or equal to 14 centimeters, greater than or equal to 16 centimeters, greater than or equal to 18 centimeters, or greater than or equal to 20 centimeters. In certain embodiments, the second dimension is less than or equal to 25 centimeters, less than or equal to 20 centimeters, less than or equal to 18 centimeters, less than or equal to 16 centimeters, less than or equal to 14 centimeters, or less than or equal to 12 centimeters. Combinations of the above recited ranges are possible (e.g., the second dimension is greater than or equal to 10 centimeters and less than or equal to 25 centimeters, the second dimension is greater than or equal to 14 centimeters and less than or equal to 16 centimeters). Other ranges are also possible.

The first layer and the second layer may have any of a variety of suitable shapes. FIG. 3B shows a top-view schematic diagram of article 101d comprising a first layer and a second layer comprising a serpentine shape, in accordance with certain embodiments.

In certain embodiments, the first layer and the second layer comprising the serpentine shape have a first dimension (e.g., a length) and a second dimension (e.g., a width) perpendicular to the first dimension. Referring, for example, to FIG. 3B, the first layer (hidden from view in FIG. 3B by second layer 105) and second layer 105 comprising the serpentine shape have first dimension 113 (e.g., length), which may be the same as first dimension 113 of article 101c shown in FIG. 3A (e.g., greater than or equal to 2 centimeters and less than or equal to 20 centimeters). In some embodiments, referring to FIG. 3B, the first layer (hidden from view in FIG. 3B by second layer 105) and second layer 105 comprising the serpentine shape have second dimension 115 (e.g., width), which may be the same as second dimension 115 of article 101c shown in FIG. 3A (greater than or equal to 10 centimeters and less than or equal to 25 centimeters).

Although the figures show an article comprising a serpentine shape, any of a variety of suitable shapes are possible. In certain embodiments, for example, the article comprises a lightning bolt shape, a half moon shape, a crescent moon shape, a gibbous moon shape, a heart shape, a three-or four-leaf clover shape, and/or a star shape. Other shapes are also possible as the disclosure is not meant to be limiting in this regard. In certain embodiments, at least two articles have complementary, nested, and/or repeating shapes.

According to some embodiments, the shape of the article is advantageously chosen and/or designed by a user, as the user may cut the article (e.g., using scissors) to provide a desired shape. For example, in certain embodiments, the article is trim-to-size. In certain embodiments, the article is packaged with one or more tracing designs configured to facilitate customization of the article by the user.

In certain embodiments wherein the article comprises a third layer (e.g., a release liner), the third layer has the same dimensions as the first layer and the second layer. In other embodiments wherein the article comprises a third layer, the third layer has one or more different dimensions than the first layer and the second layer. FIG. 4A shows a top-view schematic diagram of article 101e comprising third layer 107, in accordance with certain embodiments. As shown in FIG. 4A, third layer 107 has first dimension 113′ and second dimension 115′. In certain embodiments, first dimension 113′ of third layer 107 is greater than first dimension 113 of the first layer (hidden from view in FIG. 4A by second layer 105) and second layer 105 (e.g., 1% greater, 5% greater, 10% greater, etc.). In certain embodiments, second dimension 115′ of third layer 107 is greater than second dimension 115 of the first layer (hidden from view in FIG. 4A by second layer 105) and second layer 105 (e.g., 1% greater, 5% greater, 10% greater, etc.). FIG. 4B shows a top-view schematic diagram of an article comprising a first layer and a second layer comprising a serpentine shape, wherein the article further comprises a third layer, in accordance with certain embodiments. As shown in FIG. 4B, third layer 107 has first dimension 113′ and second dimension 115′. In certain embodiments, first dimension 113′ of third layer 107 is greater than first dimension 113 of the first layer (hidden from view by FIG. 4B by second layer 105) and second layer 105 comprising a serpentine shape (e.g., 1% greater, 5% greater, 10% greater, etc.). In certain embodiments, second dimension 115′ of third layer 107 is greater than second dimension 115 of the first layer (hidden from view in FIG. 4B by second layer 105) and second layer 105 (e.g., 1% greater, 5% greater, 10% greater, etc.).

Any of a variety of suitable production methods may be utilized to manufacture the decorative scar cover product. In some embodiments, for example, the decorative scar cover product is manufactured by mixing silicone with one or more pigments and curing the mixture. In certain embodiments, the decorative scar cover product is manufactured by injecting one or more pigments into silicone and curing the mixture.

In certain embodiments, a first layer of a decorative scar cover product comprising an adhesive silicone is manufactured using adhesive silicone (optionally mixed with one or more pigments). In some embodiments, a second layer of a decorative scar cover product comprising a non-adhesive silicone is manufactured using non-adhesive silicone mixed with one or more pigments. According to some embodiments, the second layer of the decorative scar cover product comprising the non-adhesive silicone and one or more pigments is coated onto the first layer of the decorative scar cover product comprising the adhesive silicone (and optionally one or more pigments) and cured. Any of a variety of suitable coating mechanisms may be utilized, including, for example, a commercial-grade coater. Other methods of manufacturing the decorative scar cover are also possible as the disclosure is not meant to be limiting in this regard.

According to some embodiments wherein the decorative scar cover product is disposed on a release liner, the first layer of the decorative scar cover product comprising the adhesive silicone (and optionally one or more pigments) is coated onto a third layer (e.g., the release liner) and cured. In certain embodiments, the second layer of the decorative scar cover product comprising the non-adhesive silicone and one or more pigments is coated onto the first layer of the decorative scar cover product comprising the adhesive silicone (and optionally one or more pigments) disposed on the third layer (e.g., the release liner) and cured. In other embodiments, the second layer of the decorative scar cover product comprising the non-adhesive silicone and one or more pigments is disposed (e.g., coated) on the first layer of the decorative scar cover product comprising the adhesive silicone (and optionally one or more pigments), which is then coated onto the third layer (e.g., the release liner) and cured. Any of a variety of suitable coating mechanisms may be utilized, including, for example, a commercial-grade coater. Other methods of manufacturing the decorative scar cover disposed on the release liner are also possible as the disclosure is not meant to be limiting in this regard.

As described herein, the decorative scar cover product may in the form of a sheet. Other forms are also possible, however, as the disclosure is not meant to be limiting in this regard. In certain embodiments, for example, the decorative scar cover product is in the form of a gel. In some embodiments, the gel comprises silicone (e.g., medical-grade silicone) and one or more pigments (e.g., cosmetic-grade pigments).

According to certain embodiments, the decorative scar cover product comprises one or more additives. Suitable additives include, for example, one or more binders and/or one or more therapeutic agents. Other additives are also possible as the disclosure is not meant to be limiting in this regard.

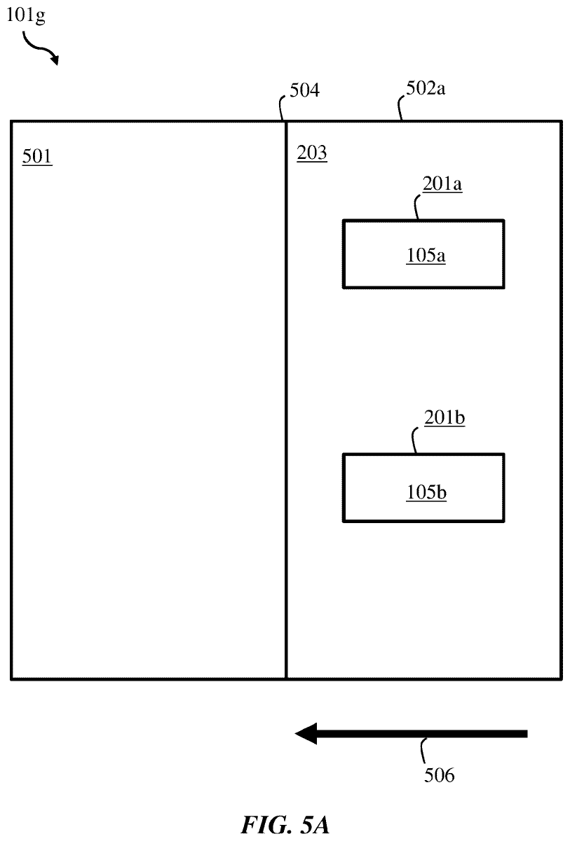

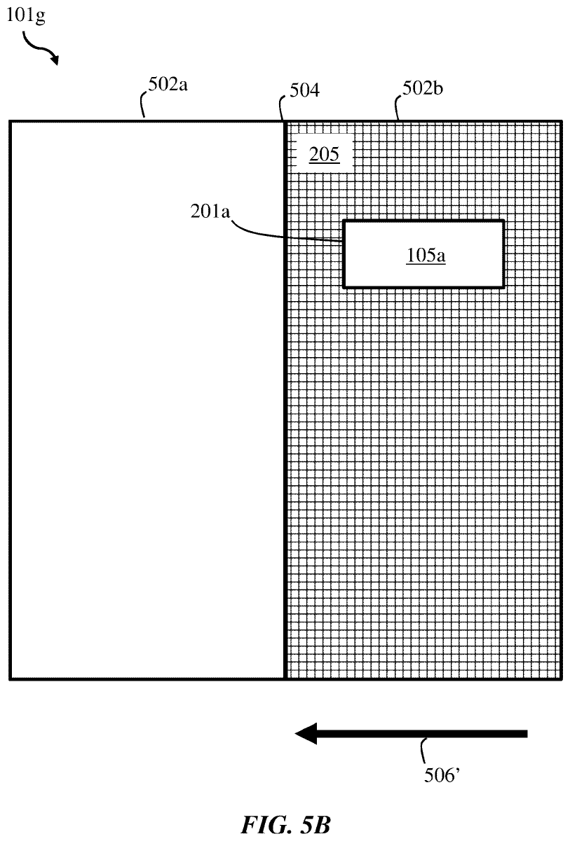

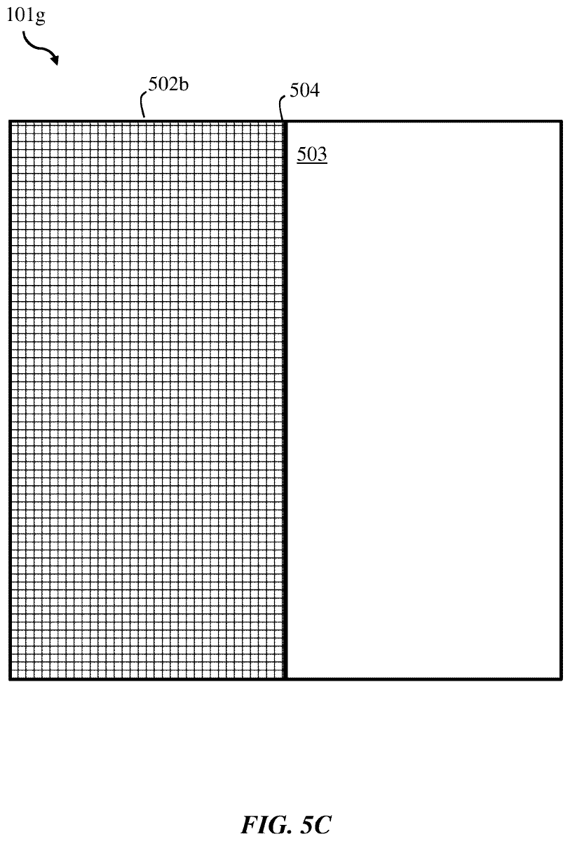

According to certain embodiments, an article comprises a first (e.g., front) cover, a second (e.g., back) cover, and a plurality of pages between the first cover and the second cover. FIGS. 5A-5C show perspective-view schematic diagrams of article 101g comprising first cover 501, second cover 503, and a plurality of pages 502 (e.g., 502a and 502b) between first cover 501 and second cover 503, in accordance with certain embodiments. According to certain embodiments, article 101g is a catalogue (e.g., a catalogue of one or more decorative scar cover products).

The first cover, second cover, and the plurality of pages may comprise any of a variety of suitable materials. In certain embodiments, for example, the first cover, the second cover, and/or the plurality of pages comprise paper (e.g., cardboard) and/or a plastic. In certain embodiments, the first cover, second cover, and/or the plurality of pages include any of the materials described herein with respect to the third layer (e.g., release liner). Other materials are also possible as the disclosure is not meant to be limiting in this regard.

According to certain embodiments, at least one first page of the plurality of pages comprises at least one decorative scar cover product disposed on a release liner.

Referring, for example, to FIG. 5A, first page 502a comprises decorative scar cover products 201 (e.g., 201a and 201b) disposed on release liner 203. As described herein, the decorative scar cover product comprises a first layer comprising an adhesive silicone and a second layer comprising a non-adhesive silicone disposed on the first layer, wherein the first layer and/or the second layer comprise one or more pigments. Referring, for example, to FIG. 5A, decorative scar cover products 201 (e.g., 201a and 201b) comprise a first layer (hidden from view in FIG. 5A by second layer 105) comprising an adhesive silicone and second layer 105 (e.g., 105a and 105b) comprising a non-adhesive silicone disposed on the first layer, wherein the first layer and/or second layer 105 (e.g., 105a and 105b) comprise one or more pigments.

As described herein in greater detail, the first layer of the decorative scar cover product comprising the adhesive silicone may be disposed on the release liner such that the adhesive silicone contacts the release liner. In certain embodiments, the first layer of the decorative scar cover product comprising the adhesive silicone is releasably adhered to the release liner. Referring, for example to FIG. 5A, the first layer (hidden from view in FIG. 5A by second layer 105) of decorative scar cover products 201 (e.g., 201a and 201b) may be releasably adhered to release liner 203. According to some embodiments, the decorative scar cover product is released (e.g., peeled) from the release liner by the hand of a user.

According to some embodiments, the first page is configured to be turned (e.g., by the hand of a user). Referring, for example, to FIG. 5A, page 502a is configured to be turned (represented in FIG. 5A by direction 506).

According to certain embodiments, at least one second page of the plurality of pages comprises a storage mechanism configured to store the decorative scar cover product (e.g., after the decorative scar cover product has been released from the release liner, applied to a scar site, and removed from the scar site). Referring, for example, to FIG. 5B, second page 502b comprises storage mechanism 205 configured to store decorative scar cover product 201a (e.g., after decorative scar cover product 201a has been removed from release liner 203 shown in FIG. 5A, applied to a scar site, and removed from the scar site). In certain embodiments, the storage mechanism is a drying rack. In certain embodiments, the storage mechanism comprises one or more grooves and/or channels in the page configured to receive the decorative scar cover product.

According to some embodiments, the second page is configured to be turned (e.g., by the hand of a user). Referring, for example, to FIG. 5B, page 502b is configured to be turned (represented in FIG. 5B by direction 506′).

The article (e.g., catalogue) may comprise any of a variety of suitable number of pages. In certain embodiments, for example, the article comprises greater than or equal to 2 pages, greater than or equal to 5 pages, or greater than or equal to 10 pages. In some embodiments, the article comprises less than or equal to 50 pages, less than or equal to 10 pages, or less than or equal to 5 pages. Combinations of the above recited ranges are possible (e.g., the article comprises greater than or equal to 2 pages and less than or equal to 50 pages, the article comprises greater than or equal to 5 pages and less than or equal to 10 pages). Other ranges are also possible. In certain embodiments, at least one first page comprises at least one decorative scar cover product and at least one second page comprises a storage mechanism configured to store the at least one decorative scar cover product.

The article (e.g., catalogue) may comprise any of a variety of suitable number of decorative scar cover products. In certain embodiments, for example, the article comprises greater than or equal to 2 decorative scar cover products, greater than or equal to 5 decorative scar cover products, or greater than or equal to 10 decorative scar cover products. In some embodiments, the article comprises less than or equal to 50 decorative scar cover products, less than or equal to 10 decorative scar cover products, or less than or equal to 5 decorative scar cover products. Combinations of the above recited ranges are possible (e.g., the article comprises greater than 2 decorative scar cover products and less than or equal to 50 decorative scar cover products, the article comprises greater than 5 decorative scar cover products and less than or equal to 10 decorative scar cover products). Other ranges are also possible.

The article (e.g., catalogue) may comprise a binding, in accordance with certain embodiments. Referring, for example, to FIGS. 5A-5C, article 101g comprises binding 504. The binding may be any of a variety of suitable bindings. In certain embodiments, for example, the binding is a case binding that glues each page of the plurality of pages together at the binding. In some embodiments, the binding is a spiral binding. Other bindings are also possible as the disclosure is not meant to be limiting in this regard.

The first (e.g., front) cover (e.g., first cover 501) and/or the second (e.g., back) cover (e.g., second cover 503) of the article (e.g., catalogue) may include brand and/or product information including, but not limited to, product branding, a summary of product materials, and/or directions for use.

In certain embodiments, the article (e.g., catalogue) is packaged such that the article is inserted into a structural sleeve. According to some embodiments, the structural sleeve includes brand and/or product information.

According to certain embodiments, a method of scar treatment is described. In some embodiments, the method comprises applying an adhesive (e.g., a decorative scar cover product) onto a scar site. As described herein, the adhesive may, in some embodiments, comprise a first layer comprising an adhesive silicone (and optionally one or more pigments) and a second layer comprising a non-adhesive silicone and one or more pigments, wherein the second layer is disposed on the first layer. In certain embodiments, applying the adhesive to the scar site comprises applying at least a portion of the first layer comprising the adhesive silicone to the scar site such that the adhesive silicone contacts the scar site. In some embodiments, the method comprises allowing the adhesive (e.g., the adhesive silicone of the first layer) to adhere to the scar site. In certain embodiments, the adhesive completely covers the scar site.

According to certain embodiments, at least two adhesives are applied to the scar site. In some embodiments, the at least two adhesives at least partially overlap (e.g., by greater than or equal to 0.5 centimeters, greater than or equal to 1 centimeter, greater than or equal to 2 centimeters, etc.) after being applied to the scar site. In certain embodiments, the at least two adhesives may have complementary, nested, and/or repeating shapes.

According to some embodiments, the adhesive advantageously hydrates the scar site. In certain embodiments, for example, the adhesive traps a user's natural moisture and hydrates the scar site without causing the skin to prune.

In certain embodiments, the adhesive is advantageously substantially (or completely) impermeable to one or more foreign species. In some embodiments, for example, the adhesive is substantially (or completely) impermeable to bacteria and/or dirt.

According to some embodiments, the adhesive is advantageously substantially (or completely) impermeable to ultraviolet (UV) light. In certain embodiments, for example, at least the second layer comprises one or more pigments evenly distributed throughout the second layer such that the one or more pigments render the second layer substantially (or completely) impermeably to UV light. In certain embodiments, for example, the second layer has a UV transmittance of less than or equal to 5% and greater than or equal to 0%, as described herein in greater detail.

The adhesive may be adhered to the scar site for any of a variety of suitable durations. In some embodiments, the adhesive is adhered to the scar site for greater than or equal to 1 hour, greater than or equal to 6 hours, or greater than or equal to 12 hours. In certain embodiments, the adhesive is adhered to the scar site for less than or equal to 24 hours, less than or equal to 12 hours, or less than or equal to 6 hours. Combinations of the above recited ranges are possible (e.g., the adhesive is adhered to the scar site for greater than or equal to 1 hour and less than or equal to 24 hours, the adhesive is adhered to the scar site for greater than or equal to 6 hours and less than or equal to 12 hours). Other ranges are also possible.

According to certain embodiments, the method of scar treatment comprises releasing the adhesive from a release liner before applying the adhesive to the scar site. As described herein, for example, the adhesive may be disposed on a release liner such that a first layer of the adhesive comprising an adhesive silicone is releasably adhered to the release liner. The adhesive may be released (e.g., peeled) from the release liner by the hand of a user, in certain embodiments.

According to some embodiments, the adhesive is reusable. For example, in certain embodiments, the method of scar treatment comprises removing the adhesive from the scar site, and re-applying the adhesive to the scar site. The adhesive may be reused for any of a variety of suitable durations. In certain embodiments, the adhesive is reused for greater than or equal to 2 days, greater than or equal to 5 days, greater than or equal to 1 week, greater than or equal to 2 weeks, or greater than or equal to 3 weeks. In some embodiments, the adhesive is reused for less than or equal to 4 weeks, less than or equal to 3 weeks, less than or equal to 2 weeks, less than or equal to 1 week, or less than or equal to 5 days. Combinations of the above recited ranges are possible (e.g., the adhesive is reused for greater than or equal to 2 days and less than or equal to 4 weeks, the adhesive is reused for greater than or equal to 1 week and less than or equal to 2 weeks). In some embodiments, during the period of reuse (e.g., greater than or equal to 2 days and less than or equal to 4 weeks), the adhesive is used each day for greater than or equal to 1 hour and less than or equal to 24 hours.

According to certain embodiments, the adhesive is water-resistant. In certain embodiments, the adhesive is washed with water in between uses. According to certain embodiments, the adhesive is stored in between uses. In certain embodiments, the adhesive is washed, stored, and dried on a drying rack in between uses. For example, referring to FIG. 5B, the adhesive (e.g., decorative scar cover product 201a) may be washed, stored, and dried on storage mechanism 205, which may be a drying rack. Other storage mechanisms are also possible, as the disclosure is not meant to be limiting in this regard.

The following application is incorporated herein by reference, in its entirety, for all purposes: U.S. Provisional Patent Application No. 63/462,448, filed Apr. 27, 2023, and entitled “DECORATIVE SCAR COVER PRODUCTS AND RELATED METHODS”.

The following examples are intended to illustrate certain embodiments of the present disclosure, but do not exemplify the full scope of the disclosure.

### EXAMPLE

The following example describes various decorative scar cover products.

The decorative scar cover product is premised on the idea that a user's recovery journey should be honored. For too long, the ecosystem of wound care products has deepened stigmas and reinforced the feeling that to be a patient you must also give up your pride and sense of style. For some patient communities, this idea of “pride in recovery” is inherent to the ways they already relate to their healing journey. For other communities, celebrating one's scar is a foreign concept requiring a more deliberate re-education over time.

The benefits of the decorative scar cover product are cosmetic, clinical, and/or psychological. The decorative scar cover product: (i) is clinically supported to improve scar size, color, and/or texture by making the scar flatter, softer, and/or less visible; (ii) hydrates the scar site (e.g., by trapping the user's natural moisture without causing the skin to prune) and reduces redness, itchiness, and/or discomfort; (iii) protects the scar site against the sun to avoid dangerous pigmentation; (iv) transforms the scar into inspiration for decorative ornamentation; (v) incorporates 100% medical-grade silicone; (vi) is reusable in that it can be removed, washed, and re-applied for up to twenty days; (vii) is water-resistant; (viii) is safe for sensitive skin and children ages three and up; and (ix) is made in the USA.

Suitable colors for the decorative scar cover product include gold (e.g., gold foil, kintsugi gold, rose gold), bronze (e.g., gunmetal), silver, blue (e.g., electric blue), and/or pink. The color of the decorative scar cover product can encompass a full spectrum of possibilities that the user can customize.

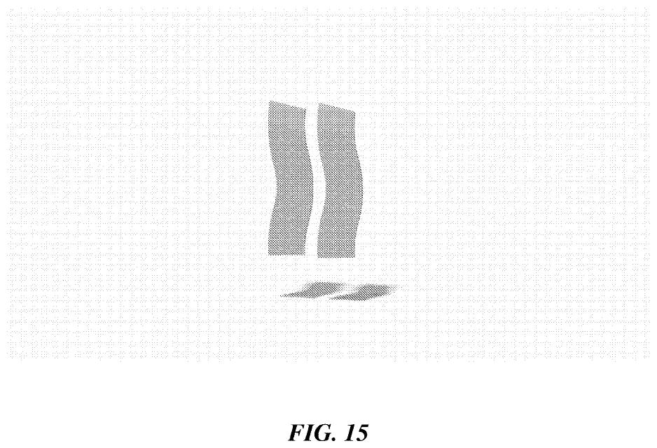

An initial assortment of decorative scar cover products features: (i) two sizes (sheets that are about 1.5 inches by about 7 inches and curved sheets that are suitable for C-section scars); (ii) three colors (kintsugi gold, silver, rose gold); and (iii) two product variations (a starter kit with a case in which to store the decorative scar cover product along with replenishment packs), thereby providing a total of eighteen different products for consumers. FIG. 15 shows a schematic diagram of a decorative scar cover product comprising a serpentine shape, in accordance with certain embodiments. FIG. 13 shows a schematic diagrams of a decorative scar cover product catalogue, in accordance with certain embodiments.

The decorative scar cover product offers significant improvements over other conventional products in that the decorative scar cover product: (i) makes the user feel beautiful and honors the user's scar versus merely covering it up; (ii) provides a physical barrier from the sun due to the inclusion of highly concentrated pigments; and (iii) embodies a self-empowering ethos not found elsewhere in the market. The decorative scar cover product is therefore positioned as a prestige beauty product that will be reinforced via pricing and distribution through influential professional and/or beauty retail channels.

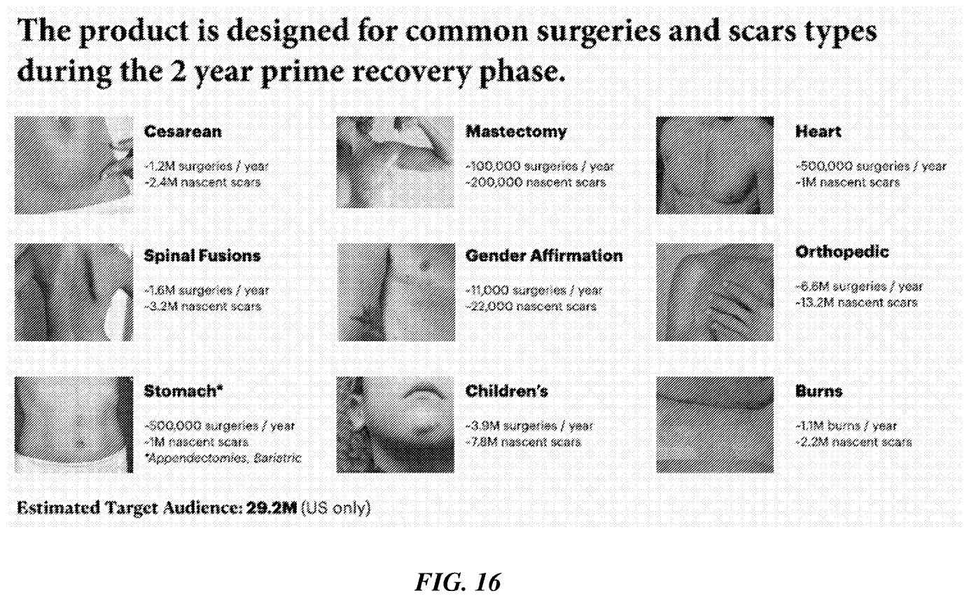

Target consumers will be women ages 25-55 and children ages 5-15. Additional consumers may be targeted via influencer cultivation to communities around gender affirmation, mastectomy, orthopedic, cardio-thoracic, and/or thyroid surgeries (see FIG. 16).

The decorative scar cover product may be distributed in a multi-channel strategy via targeted directed to consumer (DTC), select dermatology offices, select plastic surgery offices, beauty retail, and/or hospital systems. For DTC, the decorative scar cover product will aim to kindle a movement through targeted early influencer outreach to hyper-specific patient communities with an initial emphasis on gender affirmation, mastectomy, orthopedic, cardio-thoracic, and/or thyroid surgeries. Research has shown that these communities tend to be the most vocal in their appreciation for their scars as symbols of strength and personal transformation. For select dermatology and/or plastic surgery offices, pre-orders from influential practices in major metropolitan areas will be targeted via direct outreach. For beauty retail, as traction is built with targeted influential patient communities, high-end retailers will be a focus, none of which currently stock any significant assortment of scar treatment products. For hospital systems, children's hospitals will be targeted, as they are fewer in number and easier to penetrate, focusing on the mental health benefits of the product to make kids feel better about their recovery while making it easier to abide by the recommended scar treatment protocol.

Conventional scar cover products present a dull branding and packing experience that caters to an OTC drug store environment. In some embodiments described herein, a class 1 wound care product that provides a decorative scar cover designed to celebrate a user's recovery journey while protecting and healing the scar line with medical-grade silicone is described. The product draws inspiration from the Japanese art of kintsugi and momentum from concurrent trends around science-backed skincare, body art, and body positivity. The product is in the form of silicone strips sized 1.5 inches by 7 inches. The silicone strips are injected with cosmetic-grade pigments to achieve a bright color expression.

Silicone (e.g., 100% medical-grade silicone) and pigment mixtures are used in the manufacturing process to provide the decorative scar cover product. Suitable pigment mixtures include: (i) 70 wt. % DP Classy Satin Gold, 20 wt. % Diamond Sunny Gold, and 10 wt. % Diamond Crystal Gold; (ii) 70 wt. % DP Classy Satin Gold, 20 wt. % Diamond Sunny Gold, and 10 wt. % Glamour Interference Gold; (iii) 80 wt. % DP Classy Satin Gold, 10 wt. % Diamond Sunny Gold, and 10 wt. % Diamond Crystal Gold; and/or (iv) 80 wt. % DP Classy Satin Gold, 10 wt. % Diamond Sunny Gold, and 10 wt. % Glamour Interference Gold. Other pigment mixtures are also possible.

The decorative scar cover product may be manufactured according to the following steps:
(1) Tare a scale. Add the desired amount of Part A (silicone) to a beaker.
(2) Tare the scale with the beaker and silicone and add Part B (pigment mixture). Add 1 part Part B (pigment mixture) to 10 parts Part A (silicone).
(3) Mix Part A (silicone) and Part B (pigment mixture) using an appropriately sized propeller blade for 5 minutes at 450 rotations per minute (rpm).
(4) Clean the mix from the propeller blade into the beaker.
(5) Mix for another 5 minutes between 250-450 rpm to prevent emergence of air pockets. If air pockets occur, allow additional time for the batch to settle or pass the batch through a vacuum for three minutes at 28 in Hg.
(6) Scrape the mix from the propeller blade into a sterile petri dish and allow the mix to sit at room temperature to solidify. Repeat for all different pigment mixtures. Evaluate for luminosity.

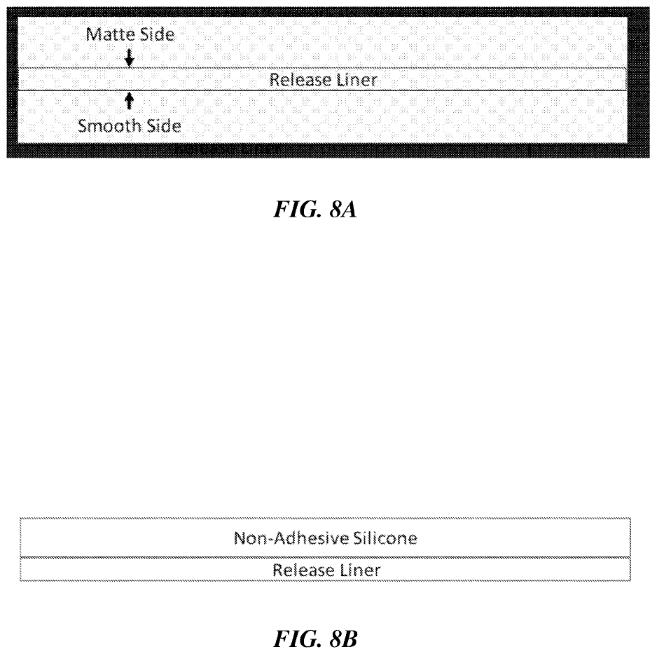

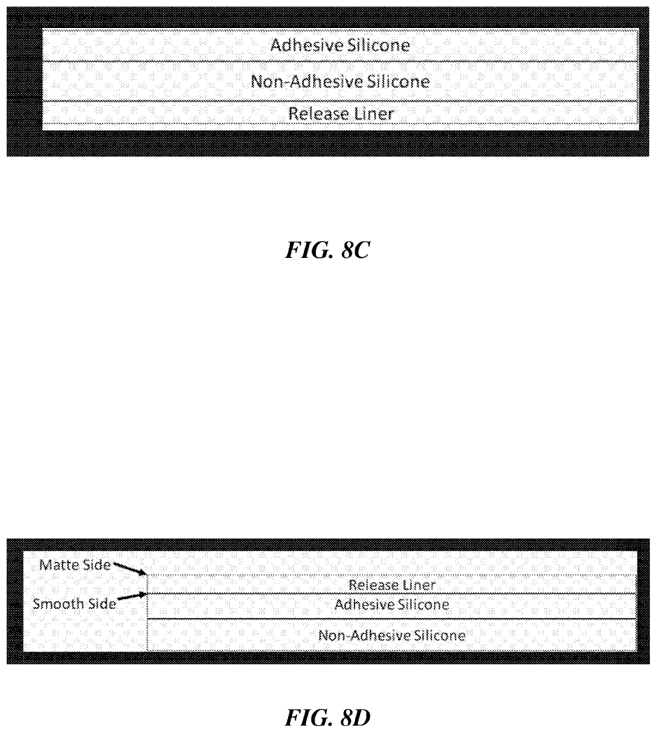

The decorative scar cover product may be applied to a release liner and cured according to the following steps:
(1) String a coater with a release liner, matte side up (see FIG. 8A).
(2) Apply the non-adhesive silicone (first layer) to the matte side, run through the curing ovens, and rewind into a roll at the tail end of the line (see FIG. 8B).
(3) After the product is completely cured, string into the front end of the coater and apply the adhesive silicone (second layer) on top of the first layer and rewind (see FIG. 8C).
(4) Allow for post-curing and the product is ready to be cut (see FIG. 8D).

The decorative scar cover product may be packaged according to the following procedure.

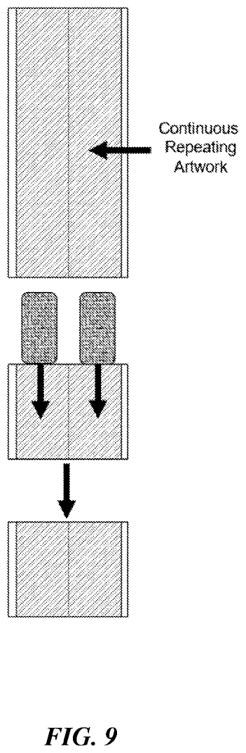

Two streams of hot seal paper (HSP) (one printed, one blank) are fed into a machine. The “H-shaped” die seals these together which seals the last package loaded and creates the bottom for the next package to receive the product. The machine is then cycled, and the following steps occur:
(1) The machine advances the HSP (e.g., 4 inches or 8 inches for longer sizes).
(2) The dies close seal the top of the package just loaded and create the bottom of the next packet to receive the product.
(3) The last product sealed is cut as 8-inch-long packets (see FIG. 9).

The decorative scar cover product manufacturing and/or packaging steps may occur in an ISO 13485 certified facility (i.e., quality management specifically designed for medical devices).

The decorative scar cover product may be provided with directions for use.

Suitable directions for use include the following steps:
(1) Wash and dry existing scar or newly healed wound area. DO NOT USE ON OPEN WOUNDS.
(2) Tear the paper packet and remove the decorative scar cover product.
(3) Trim to fit, making sure to leave 0.5 centimeters of material on each side of the scar.
(4) Peel away the protective liner from the adhesive silicone gel.
(5) Apply the decorative scar product with the adhesive silicone gel side down, toward the scar, completely covering the scar area. For best results, wear each day for a minimum of 12 continuous hours.
(6) Remove, wash, rinse, and dry the decorative scar cover product daily. Reapply as needed. Replace the decorative scar cover product currently in use when it fails to re-adhere to the skin.

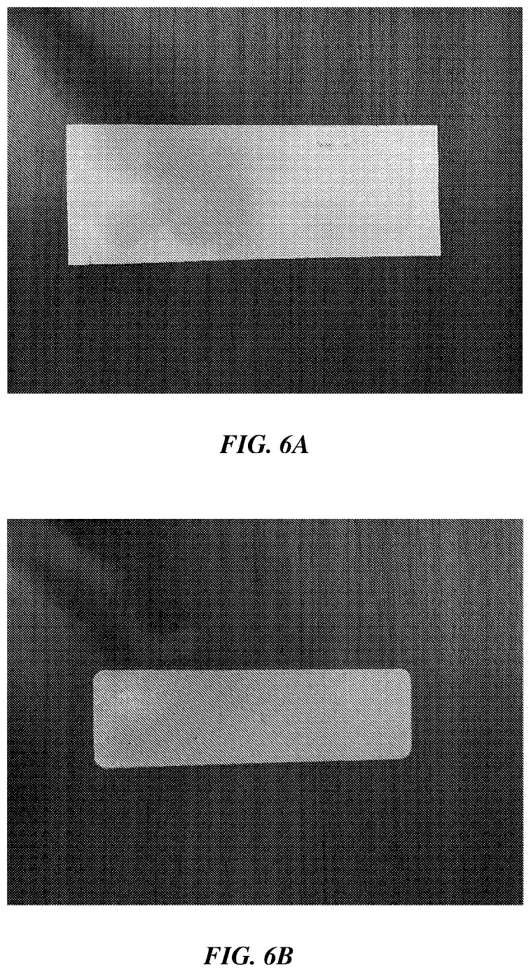

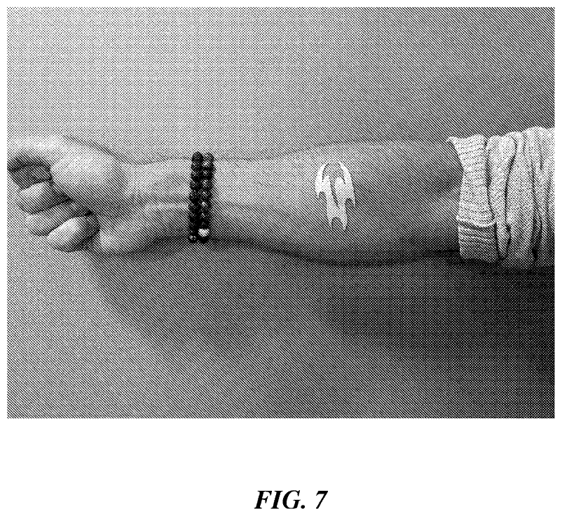

FIGS. 6A-7 show various scar cover product images. FIG. 6A shows a scar cover product in sanitary wrapping, FIG. 6B shows a scar cover product on a release liner, and FIG. 7 shows an in-situ image of a scar cover product on healing scar of a user.

In summary, described herein are trim-to-size decorative scar therapy sheets designed to celebrate the user's recovery journey while healing the underlying scar line. The product represents beauty, resilience, and confidence. Conventional wound care products are dull and designed to conceal a user's scar, while the decorative scar cover product described herein embraces a quote by the thirteenth century poet Rumi, “the wound is the place where the light enters you.” The decorative scar cover product makes a user's scar line a thing of beauty and strength. Furthermore, silicone sheeting has become a standard of care for scar therapy over the last twenty years and an utterly commodified product in which many drugstore brands compete for shelf space touting marginal differences in efficacy. The decorative scar cover product described herein, however, recognizes that the category is disconnected from the culture: in a world of body positivity and body art, scar treatment needs to catch up.

The decorative scar cover product may be in the form of a sheet for use on a user's body, joint, face, and/or other active and/or non-active site. Gels and/or sprays comprising one or more pigments mixed into silicone are also contemplated. In some embodiments, the decorative scar cover product may be customizable such that a user can take a photograph of the scar and use computer vision to translate a decorative scar cover product into a cut file that is then custom cut to fit the exact geometry of the user's scar line.

No other product in the wound care category is: (i) purposefully designed to celebrate the user and the story their scar tells; and/or (ii) is poised to diligently build a movement around scar positivity among influencers in various patient verticals. The scar cover product described herein is uniquely designed to show off a user's strength and celebrate their scar story.

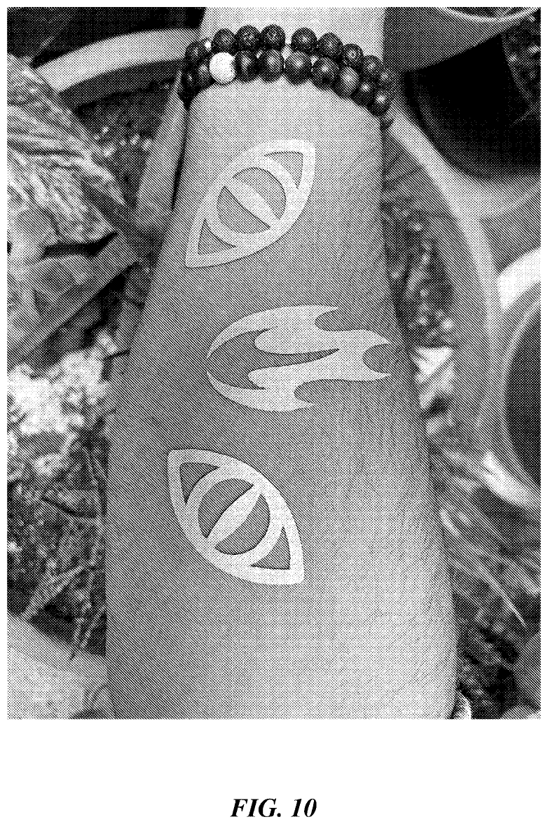

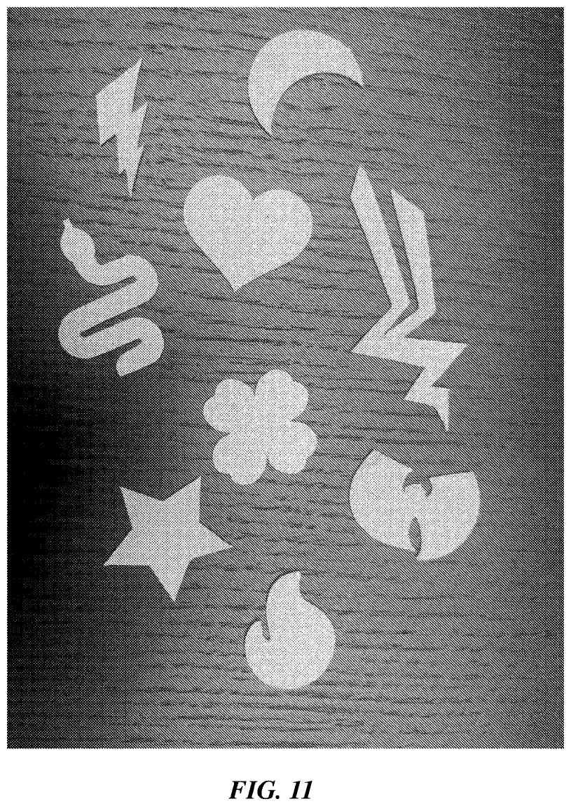

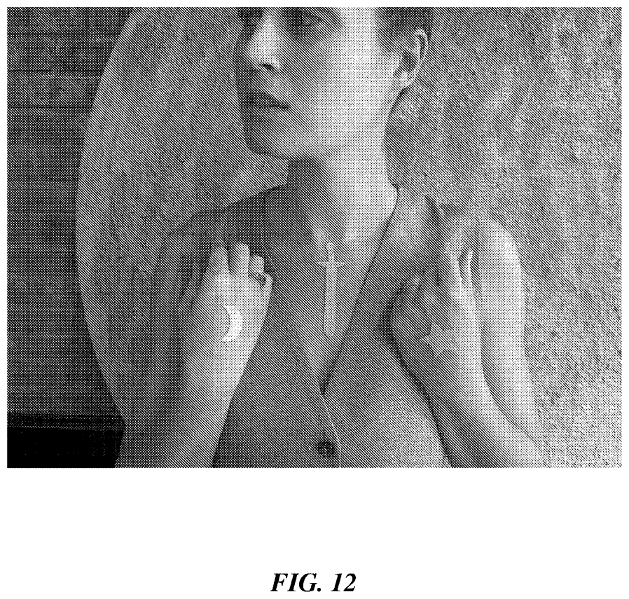

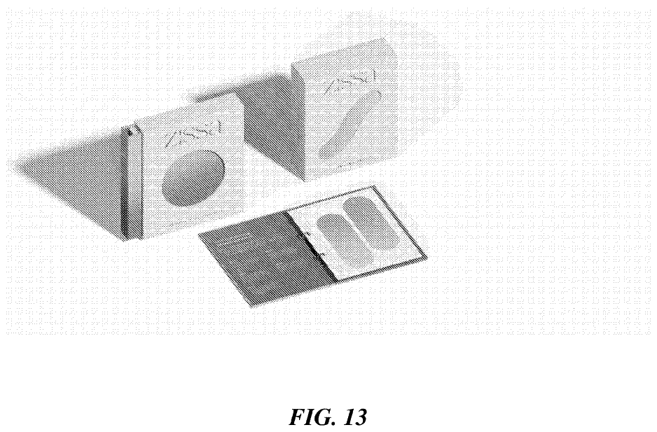

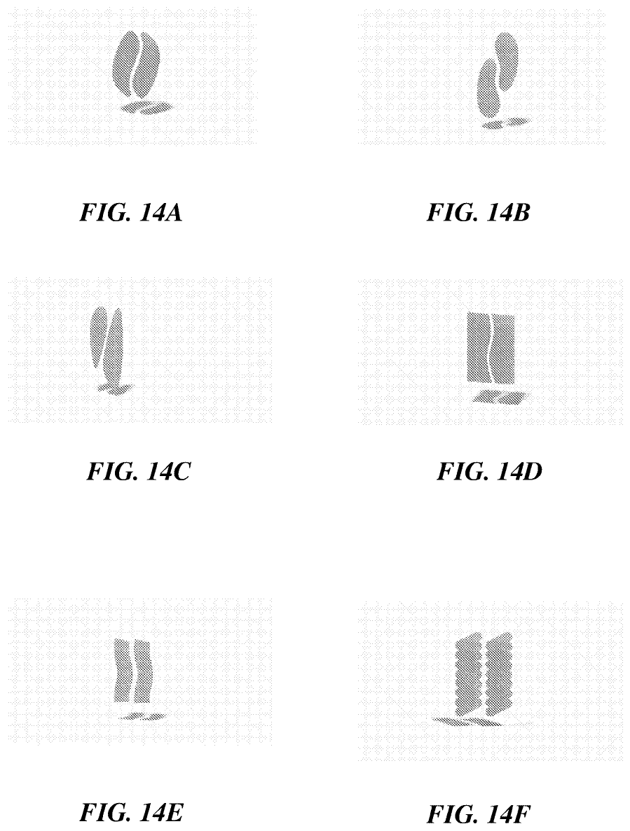

Functionally, the scar cover product is self-adhering and can be removed without inflicting trauma on the scar. The scar cover product is comfortable, non-toxic, non-allergenic, impermeable to bacteria, and provides UV protection. When is use, the scar cover product softens and flattens scar texture, hydrates and protects the scar, and reduces redness, itchiness, and discomfort, thereby visibly improving the appearance of new and old scars. Such results can be observed in as little as four to eight weeks. The scar cover product may be reusable (e.g., for up to twenty days). Suitable uses include the treatment and prevention of scars including hypertrophic and keloids resulting from injuries, burns, surgeries, and the like. Furthermore, users can cut and fashion the scar cover product into custom shapes with regular household scissors. The decorative scar cover product is a tangible symbol of strength and resilience that provides a user with the ability to feel beautiful while the user is healing. FIGS. 10-12 show various images of scar cover products. FIG. 10 shows an in-situ image of three scar cover products on the skin of a user, FIG. 11 shows an image of various scar cover product shapes and sizes, and FIG. 12 shows an in-situ image of three different scar products on the skin of a user. FIGS. 14A-14F show schematic diagrams of decorative scar cover products having complementary, nested, and/or repeating shapes.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt. %” is an abbreviation of weight percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article, comprising:
a first layer comprising an adhesive silicone; and
a second layer comprising a non-adhesive silicone disposed on the first layer, wherein the second layer comprise one or more pigments such that the second layer is impermeable to ultraviolet (UV) light.

2. The article of claim 1, wherein the first layer comprises the one or more pigments.

3. The article of claim 2, wherein the first layer comprises the one or more pigments in an amount greater than or equal to 1 weight percent (wt. %) and less than or equal to 20 wt. % versus a total weight of the first layer.

4. The article of claim 1, wherein the second layer comprises the one or more pigments such that the one or more pigments are evenly distributed throughout a bulk of the second layer.

5. The article of claim 1, wherein the second layer comprises the one or more pigments in an amount greater than or equal to 1 wt. % and less than or equal to 20 wt. % versus a total weight of the second layer.

6. The article of claim 1, further comprising a third layer, wherein the first layer comprising the adhesive silicone is disposed on the third layer such that the adhesive silicone contacts the third layer.

7. The article of claim 6, wherein the first layer and the second layer are configured to be released from the third layer.

8. The article of claim 6, wherein the third layer is a release liner.

9. The article of claim 1, wherein the first layer and the second layer have a first dimension greater than or equal to 2 centimeters and less than or equal to 20 centimeters.

10. The article of claim 9, wherein the first layer and the second layer have a second dimension greater than or equal to 10 centimeters and less than or equal to 25 centimeters, wherein the second dimension is perpendicular to the first dimension.

11. The article of claim 1, wherein the adhesive silicone and/or the non-adhesive silicone is medical-grade silicone.

12. The article of claim 1, wherein the one or more pigments are cosmetic-grade pigments.

13. The article of claim 1, wherein the first layer and the second layer have a serpentine shape.

14. A method of scar treatment, comprising:
applying an adhesive to a scar site, wherein the adhesive comprises a first layer comprising an adhesive silicone and a second layer comprising a non-adhesive silicone disposed on the first layer, wherein the second layer comprises one or more pigments such that the second layer is impermeable to ultraviolet (UV) light, and wherein applying the adhesive to the scar site comprises applying at least a portion of the first layer comprising the adhesive silicone to the scar site such that the adhesive silicone contacts the scar site; and
allowing the adhesive to adhere to the scar site.

15. The method of claim 14, wherein the adhesive is water-resistant.

16. The method of claim 14, wherein the adhesive hydrates the scar site.

17. The method of claim 14, wherein the adhesive is impermeable to bacteria.

18. The method of claim 14, wherein the adhesive completely covers the scar site.

19. The method of claim 14, further comprising releasing the adhesive from a release liner before applying the adhesive to the scar site.

20. The method of claim 14, further comprising removing the adhesive from the scar site.

21. The method of claim 20, further comprising re-applying the adhesive to the scar site.

22. An article, comprising:
a first cover and a second cover; and
a plurality of pages between the first cover and the second cover;
wherein at least one first page of the plurality of pages comprises at least one decorative scar cover product disposed on a release liner, wherein the at least one decorative scar cover product comprises a first layer comprising an adhesive silicone and a second layer comprising a non-adhesive silicone disposed on the first layer, and wherein the first layer and/or the second layer comprise one or more pigments, and
wherein at least one second page of the plurality of pages comprises a storage mechanism configured to store the at least one decorative scar cover product.

23. The article of claim 22, wherein the first layer comprising the adhesive silicone is disposed on the release liner such that the adhesive silicone contacts the release liner.

24. The article of claim 22, wherein the storage mechanism is a drying rack.

25. The article of claim 22, wherein the article is a catalogue of decorative scar cover products.
